(19) United States
(12) Patent Application Publication
(10) Pub. No.: US 2011/0017594 A1
(43) Pub. Date: Jan. 27, 2011

(54) ANALYTE SENSOR FABRICATION

(75) Inventors: James R. Petisce, San Clemente, CA (US); Wei Huang, Laguna Hills, CA (US)

Correspondence Address: EDWARDS LIFESCIENCES CORPORATION, LEGAL DEPARTMENT, ONE EDWARDS WAY, IRVINE, CA, 92614, US

(73) Assignee: Edwards Lifesciences Corporation, Irvine, CA (US)

(21) Appl. No.: 12/824,054

(22) Filed: Jun. 25, 2010

Related U.S. Application Data
(60) Provisional application No. 61/221,948, filed on Jun. 30, 2009.

(51) Int. Cl. G01N 33/50; B41F 33/00; B41F 17/00

(52) U.S. Cl. 204/403.06; 204/403.01; 101/483; 101/35

(57) ABSTRACT

A biosensor fabrication process comprising transferring one or more layers (e.g., conductive ink and/or membrane layers) from a surface to the biosensor substrate and biosensors produced therefrom. Layers are transferred from the surface to the biosensor substrate using a deposition device, such as a pick and place machine or a printing pad/plate.

### CROSS-REFERENCE TO RELATED APPLICATIONS

This application claims the benefit of U.S. Provisional Application No. 61/222,425, filed Jul. 1, 2009, which is incorporated by reference in its entirety.

### BACKGROUND

1. Field of the Invention.

The invention relates generally to fabrication methods for analyte monitoring or detecting devices and systems for fabrication same.

2. Description of Related Art.

Controlling blood glucose levels for diabetics and other patients can be a vital component in critical care, particularly in an intensive care unit (ICU), operating room (OR), or emergency room (ER) setting where time and accuracy are essential. Presently, one of the most commonly used ways to obtain a blood glucose measurement from a patient is by a direct time-point method, which is an invasive method that involves drawing a blood sample and sending it off for laboratory analysis. This time-consuming method is often incapable of producing needed results in a timely manner. Other minimally invasive methods such as finger-stick methods involve the use of a lancet or pin to pierce the skin to obtain a small sample of blood, which is then applied to a test strip and analyzed by a glucose meter. While these minimally invasive methods may be effective in determining trends in blood glucose concentration, they generally do not track glucose accurately enough to be used for intensive insulin therapy, for example, where inaccuracy at conditions of hypoglycemia could pose a very high risk to the patient.

Electrochemical analyte sensors have been developed for measuring various analytes in a substance, such as glucose. An analyte is a substance or chemical constituent that is determined in an analytical procedure, such as a titration. For instance, in an immunoassay, the analyte may be the ligand or the binder, where in blood glucose testing, the analyte is glucose. Electro-chemical analyte sensors comprise electrolytic cells including electrodes used to measure an analyte. Two types of electrochemical analyte sensors are potentiometric and amperometric analyte sensors.

Amperometric analyte sensors, for example, are known in the medical industry for analyzing blood chemistry. These types of sensors contain enzyme electrodes, which typically include an oxidase enzyme, such as glucose oxidase, that is immobilized behind a membrane on the surface of an electrode. In the presence of blood, the membrane selectively passes an analyte of interest, e.g. glucose, to the oxidase enzyme where it undergoes oxidation or reduction, e.g. the reduction of oxygen to hydrogen peroxide. Amperometric analyte sensors function by producing an electric current when a potential sufficient to sustain the reaction is applied between two electrodes in the presence of the reactants. For example, in the reaction of glucose and glucose oxidase, the hydrogen peroxide reaction product is subsequently oxidized by electron transfer to an electrode. The resulting flow of electrical current in the electrode is indicative of the concentration of the analyte of interest.

Manufacture of an analyte sensor can be problematic. To achieve accurate analyte measurement, close tolerances must be achieved during manufacture. Additionally, many analyte manufacturing techniques require a large number of steps, where each step may introduce error and/or tolerance variations. This, in turn, results in difficulty in manufacturing of large numbers of analyte sensors with high reliability and repeatability. For example, in manufacturing a biosensor, the electrodes may be formed by applying conductive inks and membrane layers manually to a circuit board. This process can be time consuming and highly variable due to the difference in application techniques from person to person.

In light of the above, systems and methods are needed to fabricate a biosensor in an accurate, reliable and efficient manner.

### SUMMARY

According to one embodiment of the present invention, a method is disclosed for fabricating a biosensor having a substrate. At least one electrode layer is deposited on a surface, the surface being releasable of the one or more layers. The one or more layers are then transferred from the surface to at least one biosensor substrate. In one embodiment, the layer(s) forms at least a portion of an electrode of the biosensor.

In combination with the above embodiment, the method may be configured according to at least one of the following: the method further includes treating the one or more layers after deposition; the transferring step is performed using a pick and place device; the one or more layers includes conductive ink, optionally in combination with an enzyme-containing layer; the step of depositing and the step of transferring are cooperative coupled together; and the one or more layers comprise at least one or more of an electrode layer, an interference layer, an enzyme layer, a flux-limiting layer, or any combination thereof.

In accordance with another embodiment of the present invention, a system to fabricate a biosensor includes a deposition device for depositing one or more layers on a surface, and a pick and place device for transferring the layers from the surface to a biosensor substrate.

In combination with the embodiment presented immediately above, the system may be configured according to at least one of the following: the system further includes a device to cut the one or more layers into one or more predetermined shapes on the surface; the one or more layers comprises conductive ink, an electrode layer, an interference layer, an enzyme layer, a flux-limiting layer, or any combination thereof; the pick and place device is adapted to automatically transfer at least a portion of the one or more layers to at least a portion of the biosensor; and the pick and place device and the deposition device are cooperatively configured.

In accordance with another embodiment of the present invention, disclosed is a method for fabricating at least one biosensor. One or more layers (e.g., conductive ink, membrane layers, etc.) are formed on a surface and a plurality of separate layer elements may be formed from such layer(s). At least one of these layer elements are transferred from the surface to a biosensor substrate.

In combination with the embodiment presently immediately above, the method may be configured according to at least one of the following: one of the layers includes conductive ink and/or membrane layers; the transferring step is performed using a pick and place device; the pick and place device transfers the at least one layer element in proximity to an electrode surface of the biosensor substrate; the method further includes pad printing one or more membrane layers to one of the layer elements;

In accordance with another embodiment of the present invention, disclosed is a system for fabricating at least one biosensor. The system includes a conductive ink deposition unit for depositing the conductive ink on a surface, a device for forming a plurality of conductive ink elements on the surface and a pick and place device for transferring at least one of the plurality of conductive ink elements from the surface to a biosensor substrate.

In accordance with another embodiment of the present invention, a method for fabricating at least one biosensor is disclosed. A position-controlled platen is provided for receiving at least one substrate, where each substrate has at least one surface area portion adapted for receiving an electrode-forming material. The platen is positioned in a predetermined position relative to a deposition unit and at least one layer is deposited on a surface area portion of each substrate.

In combination with the embodiment presently immediately above, the method may be configured according to at least one of the following: adjusting the x-coordinates, y-coordinates, and/or z-coordinates of the platen position; the method further including depositing the layers at a plurality of portions on a substrate to form a plurality of electrodes on the biosensor; and the at least one layer comprises a conductive ink and at least one membrane layer.

In accordance with still yet another embodiment of the present invention, a system for fabricating at least one biosensor includes an etched printing plate sized and shaped for receiving at least one layer, and a pad for contacting the layer and for releasably transferring the layer to a biosensor substrate.

In combination with the embodiment presented immediately above, the system may be configured according to at least one of the following: the printing plate and the pad are integrated in a machine; the substrate includes an electrode portion having conductive ink; the pad is further configured to releasably transfer the at least one layer from the printing plate in proximity to the electrode portion; the at least one layer comprises a conductive ink and one or more membrane layers; and the membrane layers includes at least one of an enzyme layer, an interference layer and a flux limiting layer.

In accordance with still yet another embodiment of the present invention, a method for fabrication of a biosensor includes depositing a first working electrode, a second working electrode, a counter electrode and a reference electrode. The first working electrode is deposited by depositing a first conductive ink layer on a surface and transferring the first conductive ink layer to the substrate. The second working electrode is deposited by depositing a second conductive ink layer on the surface, depositing at least one membrane layer over the second conductive ink layer, and transferring the second conductive ink layer to the substrate. The counter electrode is deposited by depositing a third conductive ink layer on the surface and transferring the third conductive ink layer from the surface to the substrate. The reference electrode is deposited by depositing a fourth conductive ink layer on the surface and transferring the fourth conductive ink layer from the surface to the substrate. One or more of these steps may be performed using a printing pad process or a pick and place device and at least one of these steps may be automated.

### BRIEF DESCRIPTION OF THE DRAWINGS

Henceforth reference is made to the accompanied drawings and associated text, whereby the present invention is described through given examples and provided embodiments for a better understanding of the invention, wherein:

FIG. 1a is an illustrative block diagram of an analyte monitoring system according to one embodiment of the present invention;

FIG. 1b is a schematic diagram of a four-electrode biosensor of an analyte system in accordance with one embodiment of the present invention;

FIG. 1c is a schematic diagram of a four-electrode biosensor of the analyte system of FIG. 1a;

FIG. 1d is a schematic diagram of a cross-sectional view of a biosensor electrode in accordance with one embodiment of the present invention;

FIG. 2 is a flow chart of a disclosed process of electrode layer(s) deposition according to one embodiment of the present invention;

FIG. 3 illustrates an exemplary system to implement the disclosed process of electrode deposition of FIG. 2 according to one embodiment of the present invention;

FIG. 4 is an exemplary system and process of electrode deposition according to another embodiment of the present invention;

FIG. 5 illustrates another system and process of electrode deposition according to yet another embodiment of the present invention;

FIG. 6 is a flow chart of a disclosed process of electrode deposition according to one embodiment of the present invention; and

FIGS. 7A-7H illustrate the disclosed process of FIG. 6 according to one embodiment of the present invention.

### DETAILED DESCRIPTION

The present invention now will be described more fully hereinafter with reference to the accompanying drawings, in which some, but not all embodiments of the invention are shown. Indeed, these embodiments may be embodied in many different forms and should not be construed as limited to the embodiments set forth herein; rather, these embodiments are provided so that this disclosure will satisfy applicable legal requirements. Like numbers refer to like elements throughout. While the foregoing description refers generally to the fabrication of a single biosensor, the fabrication process is envisaged for the partial or whole fabrication of multiple biosensors (e.g., simultaneously and/or concurrently), and as such is hereby incorporated as embodiments thereof.

Embodiments of the present invention provide systems and methods for fabrication of at least one biosensor, such as an electro-chemical biosensor or amperometric sensor. One exemplary component of the biosensor fabrication process relates to fabrication of one or more electrodes of the biosensor. In the fabrication process, the electrodes may be contacted with one or more layers. Examples of the layers include electrode layers, membrane layers, conductive ink layer and other materials/layers. Fabrication of the biosensor and electrodes may be performed using various methods and systems as disclosed herein. In one embodiment, fabrication of the biosensor includes providing a substrate, providing a conveyer or other surface, depositing one or more layers on the conveyer, and transferring the layers from the conveyer to the substrate.

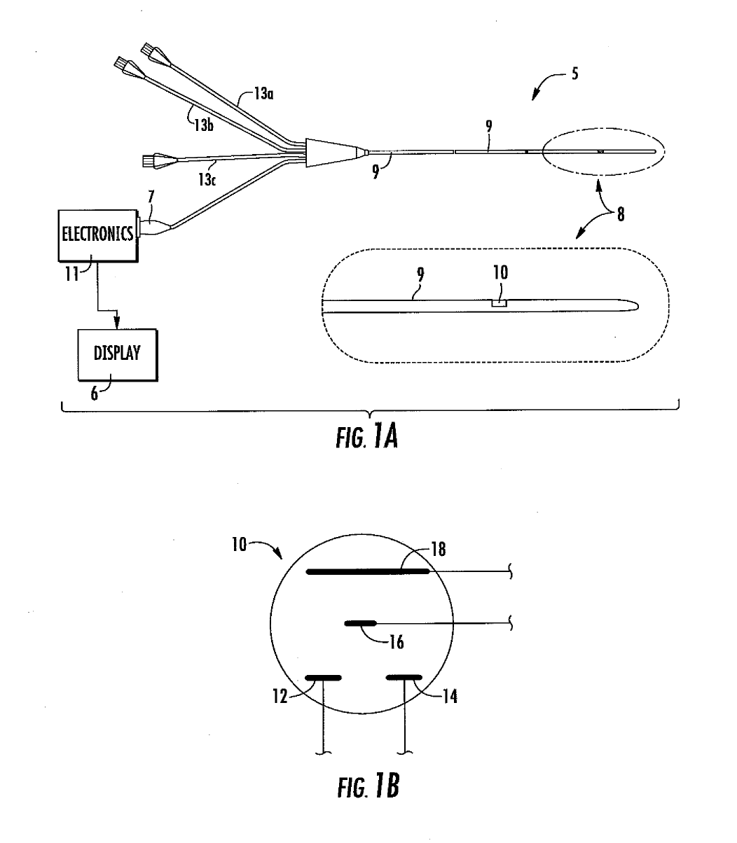

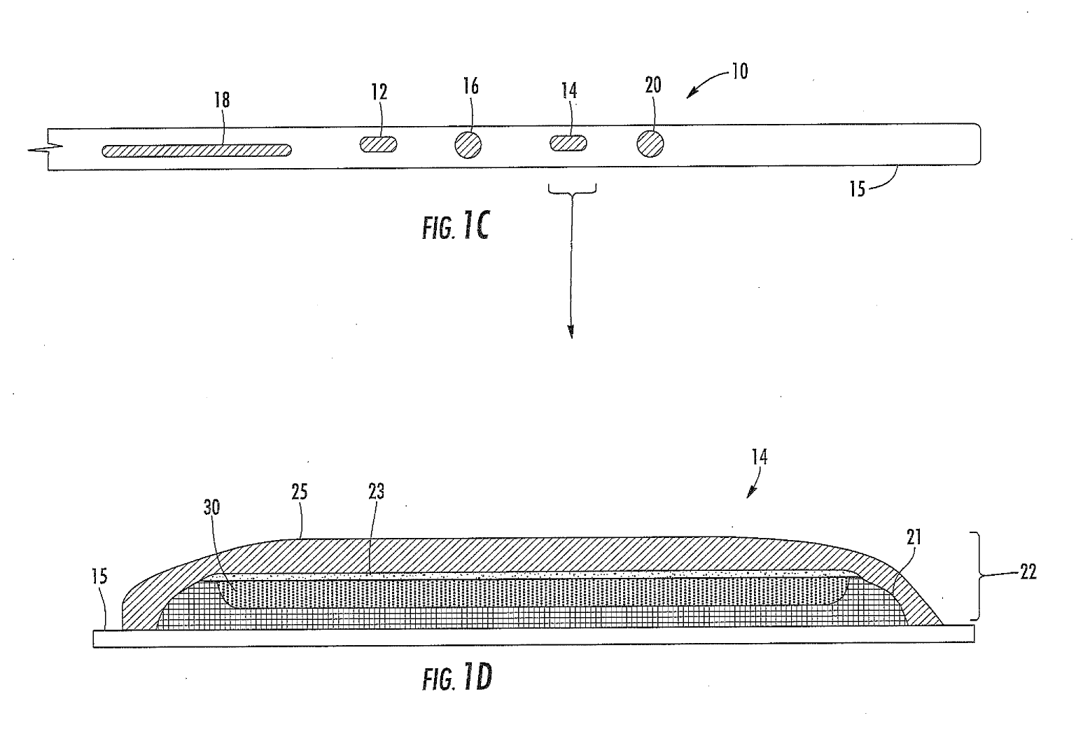

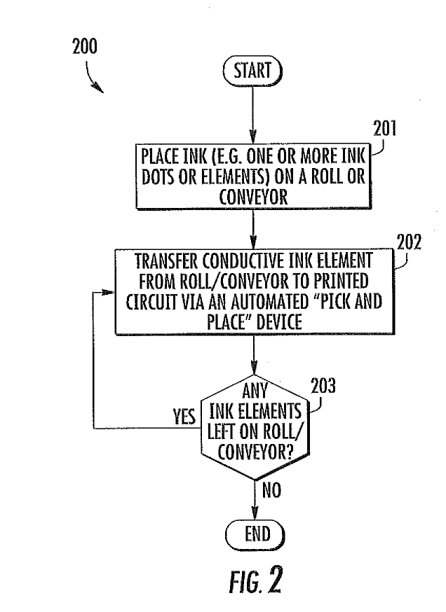

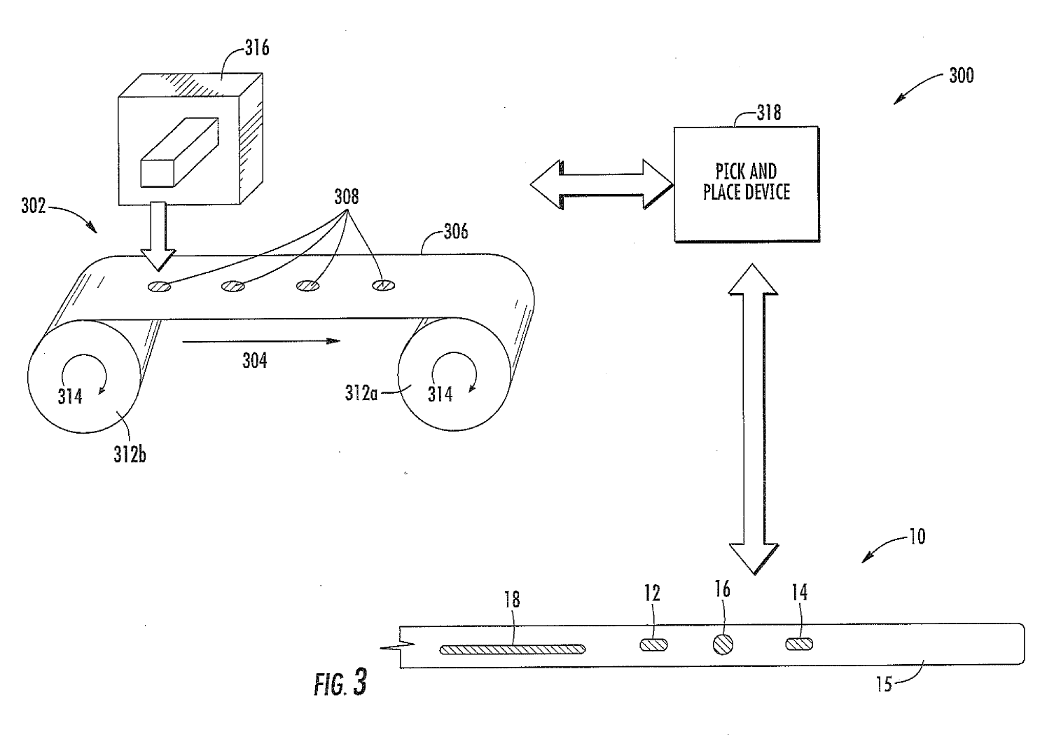

In another embodiment, the biosensor may be fabricated by coating a substrate with one or more layers and drying the layers to form a sheet of material. Thereafter, disks or strips of the layers are formed out of the sheet of material and transferred to the biosensor substrate using a pick and place machine. In an alternative embodiment, the layers may be directly deposited on the biosensor substrate by placing the biosensor substrate on a platen of a deposition machine, adjusting x and y-coordinates of the platen so that the predetermined area of the biosensor substrate to be printed with the layers is positioned below the deposition machine print head, and depositing one or more of the layers to the surface of the biosensor substrate. In yet another embodiment, fabrication of a biosensor may also include pad printing one or more layers by a process of depositing one or more precursor materials in a reservoir that feeds a printing plate and transferring the layers from the etched printing plate to the substrate with a printing pad machine or device. The above processes and systems are described in more detail later with reference to the FIGS. 1-7.

Before describing the fabrication processes, an analyte system, including an exemplary fabricated biosensor, will now be described with reference to FIGS. 1a-d. For informational purposes, FIG. 1a illustrates an exemplary analyte system 5 according to one embodiment of the present invention. The analyte system 5 includes at least: a fabricated biosensor 10, control electronics 11, an electrical connection 7, and a catheter 9. As illustrated, the biosensor 10 is fitted into a catheter 9. A window or opening may be fainted in the catheter adjacent to the location of the biosensor. In this configuration, when the catheter is inserted intravenously, the sensor is exposed to the blood stream of the patient for performing analyte concentration measurements, such as glucose concentration measurements. As illustrated, the catheter may have one or more additional lumens 13. Other medical devices may be used in conjunction with the biosensor and fabrication processes disclosed herein.

The control electronics 11 are configured to interact with the biosensor 10 so as to perform analyte concentration detection and/or monitoring. For example, the control electronics 11 may include components to provide power to the biosensor 10, as well as electronics to receive signals output from the biosensor 10. Additionally, other electronics may be provided to perform signal processing and analog to digital signal conversion. Further, yet other electronics are provided to process the signals output by the biosensor 10 to determine analyte concentration measurements and possibly present such measurements on a display 6 and/or store such measurements via a storage medium (not shown). In the illustrated embodiment, the control electronics 11 are shown as remote from the biosensor 10 and in communication with the biosensor 10 by either wiring or in some embodiments, wireless communication. In some embodiments, some or all of the control electronics 10 can be located more proximate to the biosensor 10, such as at a connector 7 of the catheter 9 (as illustrated in FIG. 1 a) or possibly resident with the biosensor 10.

The biosensor 10 is capable of measuring one or more analytes present in a liquid or chemical composition. In one embodiment, the biosensor 10 measures glucose level in vivo, for example, by contacting the biosensor 10 with circulating blood. It should be understood that various other systems (not shown) may be attached to this system, including computer systems, output devices (including a display 6), input devices, and other appropriate devices.

The systems and methods of the present invention may be used to fabricate any biosensor using a dual electrode measurement system. For example, the systems and methods may be used to fabricate electro-chemical biosensors having electrolytic cells, such as amperometric and potentiometric biosensors containing two or more electrodes used to measure an analyte in a substance, such as glucose in blood, where the analyte measurement is based on a comparison of two or more electrodes of the electrolytic cell.

For example, Figure lb is a schematic diagram of an amperometric, four-electrode biosensor 10 which can be fabricated using the systems and methods of the present invention. In the illustrated embodiment, the biosensor 10 includes two working electrodes: a first working electrode 12 and a second working or blank electrode 14. The first working electrode 12 may be a platinum based enzyme electrode, i.e. an electrode containing or immobilizing an enzyme layer. In one embodiment, the first working electrode 12 may immobilize an oxidase enzyme, such as in the sensor disclosed in U.S. Pat. No. 5,352,348, the contents of which are hereby incorporated by reference. In some embodiments, the biosensor is a glucose sensor, in which case the first working electrode 12 may immobilize a glucose oxidase enzyme. The first working electrode 12 may be formed using platinum, or a combination of platinum and carbon materials. The second working electrode 14 may be substantially identical in all respects to the first working electrode 12, except that it may not contain an enzyme layer. The biosensor 10 further includes a reference electrode 16 and a counter electrode 18. The reference electrode 16 establishes a fixed potential from which the potential of the counter electrode 18 and the working electrodes 12 and 14 may be established. The counter electrode 18 provides a working area for conducting the majority of electrons produced from the oxidation.

The amperometric biosensor 10 operates according to an amperometric measurement principle, where the first working electrode 12 is held at a positive potential relative to the reference electrode 16. In one embodiment of a glucose monitoring system, the positive potential is sufficient to sustain an oxidation reaction of hydrogen peroxide, which is the result of glucose reaction with glucose oxidase. Thus, the first working electrode 12 may function as an anode, collecting electrons produced at its surface that result from the oxidation reaction. The collected electrons flow into the first working electrode 12 as an electrical current. In one embodiment with the first working electrode 12 coated with glucose oxidase, the oxidation of glucose produces a hydrogen peroxide molecule for every molecule of glucose when the working electrode 12 is held at a potential between about +450 mV and about +650 mV. The hydrogen peroxide produced oxidizes at the surface of the first working electrode 12 according to the equation:

H₂O₂→2H⁺+O₂+2e⁻

The equation indicates that two electrons are produced for every hydrogen peroxide molecule oxidized. Thus, under certain conditions, the amount of electrical current may be proportional to the hydrogen peroxide concentration. Since one hydrogen peroxide molecule is produced for every glucose molecule oxidized at the first working electrode 12, a linear relationship exists between the blood glucose concentration and the resulting electrical current. The embodiment described above demonstrates how the first working electrode 12 may operate by promoting anodic oxidation of hydrogen peroxide at its surface. Other embodiments are possible, however, wherein the first working electrode 12 may be held at a negative potential. In this case, the electrical current produced at the first working electrode 12 may result from the reduction of oxygen. The following article provides additional information on electronic sensing theory for amperometric glucose biosensors: J. Wang, “Glucose Biosensors: 40 Years of Advances and Challenges,” Electroanaylsis, Vol. 13, No. 12, pp. 983-988 (2001).

The voltage potential provided by the reference electrode 16 is also provided to the second working or blank electrode 14. As the second working electrode 14 is substantially similar to the first working electrode 12, but for the absence of an enzyme layer, the second working electrode 14 provides an indication of conductivity of both the first and second electrodes structures. As such, by comparing the current output between the first and second working electrodes in response to the potential from the reference electrode, the effects of the enzyme layer on the first working electrode 12 output can be isolated. For example, the current output by the second working or blank electrode 14 may be subtracted from the current output from the first working electrode 12 to there by determine the effects of the enzyme layer's interaction with an analyte. This difference provides an approximation of the amount of the tested analyte in the fluid being monitored.

FIG. 1c illustrates a schematic diagram of an amperometric, four-electrode biosensor 10 according to another embodiment of the present invention. The biosensor 10 is operably coupled to system 5 of Figure la. The biosensor 10 of FIG. 1c includes a first working electrode 12, a second working electrode 14, a blank electrode 16 and a counter electrode 18 and functions similar to the biosensor of FIG. 1b, as previously described.

The biosensor 10 of this embodiment further includes a temperature sensor 20, such as a thermo-couple. The temperature sensor may be located on either the same side or an opposite side of the substrate from the electrodes. In some embodiments, the temperature sensor is used to monitor the temperature surrounding the sensor. Note that traces connecting electronics to each electrode and the temperature sensor (20) are not illustrated in FIG. 1c.

The biosensor 10 of FIG. 1c may be fabricated on a substrate 15, such as a rigid, semi-rigid, or flexible substrate. In one aspect, the substrate is flexible to allow for the sensor to be adapted and exposed from within the lumen of a small-diameter catheter 9 (FIG. 1a) positioned in vivo.

The rigid substrate may be a semiconductor or dielectric polymeric material. Suitable semiconductor substrates may be of conventional semiconductor materials such as silicon, silicon dioxide, or gallium arsenide. Silicon substrates may have native oxide or polysilicon layer or may have silicon nitride layers. Other inorganic, semi-conductive or non-conductive materials may be used as a substrate. In one aspect, the inorganic substrate excludes borosilicate glasses as these materials are generally not readily adaptable to MEMs/IC processing. The substrate may be a flexible dielectric polymeric material selected from polyimides, polyamides, polycarbonates, polysulfones, or copolymers thereof.

FIG. 1d illustrates a cross-sectional side view of a portion of substrate in the vicinity of the working electrode 14 according to one embodiment of the present invention. As illustrated, the fabricated working electrode 14 may include conductive ink layer 21 that is at least partially coated with an analyte sensing membrane 22. The depicted analyte sensing membrane 22 includes a series of membrane layers, such as an interference layer 30, an enzyme layer 23, a flux limiting layer 25 and/or other additional layers (not shown). Each of these layers is discussed in more detail below.

### Electrode Layer

In selected aspects, the layer comprises an electrode layer (not shown). The electrode layer is provided to ensure that an electrochemical reaction occurs between the electroactive surfaces of the working electrode and the reference electrode, and thus the electrode layer is preferably situated more proximal to the electroactive surfaces than an interference and/or an enzyme layer. Preferably, the electrode layer includes a coating that provides and maintains a layer of water at the electrochemically reactive surfaces of the sensor such that an environment between the surfaces of the working electrode and the reference electrode, which facilitates an electrochemical reaction between the electrodes, is provided. The electrode layer can also assist in stabilizing the operation of the sensor by accelerating electrode start-up and drifting problems caused by inadequate electrolyte in the vicinity of the electrode surface. The material that forms the electrode layer may also provide a protective environment against pH-mediated damage that may result from electrochemical activity of the electrodes.

In some aspects, the electrode layer is a flexible, water-swellable, hydrogel film having a “dry film” thickness of from about 0.05 micron or less to about 20 microns or more, more preferably from about 0.05, 0.1, 0.15, 0.2, 0.25, 0.3, 0.35, 0.4, 0.45, 0.5, 1, 1.5, 2, 2.5, 3, or 3.5 microns to about 4, 5, 6, 7, 8, 9, 10, 11, 12, 13, 14, 15, 16, 17, 18, 19, or 19.5 microns, and more preferably still from about 2, 2.5 or 3 microns to about 3.5, 4, 4.5, or 5 microns. “Dry film” thickness refers to the thickness of a cured (dry) film fabricated by the methods described herein.

In some preferred aspects, the electrode layer is fabricated as described herein from a hydrophilic polymer such as polyvinylpyrrolidone (PVP). An electrode layer formed from PVP has been shown to reduce break-in time of analyte sensors; for example, a glucose sensor comprising a cellulosic-based interference domain.

Although an independent electrode layer is described herein, in some embodiments sufficient hydrophilicity can be provided in the interference domain and/or enzyme layer (the layer(s) adjacent to the electroactive surfaces) so as to provide for the full transport of ions in the aqueous environment (e.g. without a distinct electrode layer). In these embodiments, an electrode layer is not necessary. Preferably, the electrode layer is deposited by the methods disclosed herein as further described below.

### Interference Layer

The interference layer 30 prevents or reduces migration of chemical species through the analyte sensing membrane. Interferents may be molecules or other species that may be reduced or oxidized at the electrochemically reactive surfaces of the sensor, either directly or via an electron transfer agent, to produce a false positive analyte signal (e.g., a non-analyte-related signal). This false positive signal generally causes the subject's analyte concentration to appear higher than the true analyte concentration. For example, in a hypoglycemic situation, where the subject has ingested an interferent (e.g., acetaminophen), the artificially high glucose signal may lead the subject or health care provider to believe that they are euglycemic or, in some cases, hyperglycemic. As a result, the subject or health care provider may make inappropriate or incorrect treatment decisions. In one aspect, the interference layer substantially restricts or eliminates the passage therethrough of one or more interfering species. Interfering species for a glucose sensor include, for example, acetaminophen, ascorbic acid, bilirubin, cholesterol, creatinine, dopamine, ephedrine, ibuprofen, L-dopa, methyl dopa, salicylate, tetracycline, tolazamide, tolbutamide, triglycerides, urea and uric acid. The interference layer may be less permeable to one or more of the interfering species than to a target analyte species.

In one aspect, the interference layer is formed from a material selected from cellulose ester derivatives, silicones, polytetrafluoroethylene, polyethylene-co-tetrafluoroethylene, polyolefin, polyester, polycarbonate, biostable polytetrafluoroethylene, homopolymers, copolymers, terpolymers of polyurethanes, polypropylene (PP), polyvinylchloride (PVC), polyvinylidene fluoride (PVDF), polybutylene terephthalate (PBT), polymethylmethacrylate (PMMA), polyether ether ketone (PEEK), polyurethanes, cellulosic polymers, polysulfones, tetrafluoroethylene-perfluoro-3,6-dioxa-4-methyl-7-octenesulfonic acid copolymer (Nafion) and block copolymers thereof including, for example, di-block, tri-block, alternating, random and graft copolymers. Combinations of the above polymers may be used. In one aspect, the interference layer is selected from one or more cellulosic derivatives. Additionally, mixed ester cellulosic derivatives may be used, for example, cellulose acetate butyrate, cellulose acetate phthalate, cellulose acetate propionate, cellulose acetate trimellitate, as well as their copolymers and terpolymers, with other cellulosic or non-cellulosic monomers, including cross-linked variations of the above. Other polymers, such as polymeric polysaccharides having similar properties to cellulosic derivatives, may be used as an interference material or in combination with the above cellulosic derivatives. Other esters of cellulose may be blended with the mixed ester cellulosic derivatives.

In another aspect, the interference layer is formed from cellulose acetate butyrate. Cellulose acetate butyrate is a cellulosic polymer having both acetyl and butyl groups, and hydroxyl groups. A cellulose acetate butyrate having about 35% or less acetyl groups, about 10% to about 25% butyryl groups, and hydroxyl groups making up the remainder may be used. A cellulose acetate butyrate having from about 25% to about 34% acetyl groups and from about 15 to about 20% butyryl groups may also be used, however, other amounts of acetyl and butyryl groups may be used. A preferred cellulose acetate butyrate contains from about 28% to about 30% acetyl groups and from about 16 to about 18% butyryl groups.

The concentration of solids in the casting solution may be adjusted to deposit a sufficient amount of solids or film on the electrode in one layer (e.g., in one dip or spray) to form a layer sufficient to block an interferant with an oxidation or reduction potential otherwise overlapping that of a measured species (e.g., H₂O₂), measured by the sensor. For example, the casting solution's percentage of solids may be adjusted such that only a single layer is required to deposit a sufficient amount to form a functional interference layer that substantially prevents or reduces the equivalent glucose signal of the interferant measured by the sensor. A sufficient amount of interference material would be an amount that substantially prevents or reduces the equivalent glucose signal of the interferant of less than about 30, 20 or 10 mg/dl. By way of example, the interference layer is preferably configured to substantially block about 30 mg/dl of an equivalent glucose signal response that otherwise would be produced by acetaminophen by a sensor without an interference layer. Such equivalent glucose signal response produced by acetaminophen would include a therapeutic dose of acetaminophen. Any number of coatings or layers formed in any order may be suitable for forming the interference layer of the sensor disclosed herein.

In one aspect, the fabrication process comprises depositing the interference layer either directly onto the etched printing plate, directly onto the biosensor substrate or both.

The interference layer may be applied to provide a thickness of from about 0.05 micron or less to about 20 microns or more, more preferably from about 0.05, 0.1, 0.15, 0.2, 0.25, 0.3, 0.35, 0.4, 0.45, 0.5, 1, 1.5, 2, 2.5, 3, or 3.5 microns to about 4, 5, 6, 7, 8, 9, 10, 11, 12, 13, 14, 15, 16, 17, 18, 19, or 19.5 microns, and more preferably still from about 1, 1.5 or 2 microns to about 2.5 or 3 microns. Thicker membranes may also be desirable in certain embodiments, but thinner membranes may be generally preferred because they generally have a lower affect on the rate of diffusion of hydrogen peroxide from the enzyme membrane to the electrodes.

### Enzyme Membrane Layer

In one embodiment, the enzyme layer 23 comprises an enzyme and a hydrophilic polymer. In one aspect, the enzyme layer comprises an enzyme deposited in the etched printing plate solely and/or directly onto at least a portion of the interference layer.

In one aspect, the enzyme layer comprises a enzyme and a hydrophilic polymer selected from poly-N-vinylpyrrolidone (PVP), poly-N-vinyl-3-ethyl-2-pyrrolidone, poly-N-vinyl-4,5-dimethyl-2-pyrrolidone, polyacrylamide, poly-N,N-dimethylacrylamide, polyvinyl alcohol, polyurethanes, polymers with pendent ionizable groups (polyelectrolytes) and copolymers and/or blends thereof. In one embodiment, the enzyme layer comprises poly-N-vinylpyrrolidone. In another embodiment, the enzyme layer comprises glucose oxidase, poly-N-vinylpyrrolidone and optionally an amount of crosslinking agent sufficient to immobilize the enzyme.

The molecular weight of the hydrophilic polymer of the enzyme layer is such that fugitive species are prevented or substantially inhibited from leaving the sensor environment and more particularly, fugitive species are prevented or substantially inhibited from leaving the enzyme's environment when the sensor is initially deployed.

The hydrophilic polymer of the enzyme layer may further include at least one protein and/or natural or synthetic material. For example, the enzyme layer may further include, for example, serum albumins, polyallylamines, polyamines and the like, as well as combination thereof

The enzyme of the enzyme layer may be immobilized in the sensor. The enzyme may be encapsulated within the hydrophilic polymer and may be cross-linked or otherwise immobilized therein. The enzyme may be cross-linked or otherwise immobilized optionally together with at least one protein and/or natural or synthetic material. In one embodiment, the hydrophilic polymer-enzyme composition comprises glucose oxidase, bovine serum albumin, and poly-N-vinylpyrrolidone. The composition may further include a cross-linking agent, for example, a dialdehyde such as glutaraldehdye, to cross-link or otherwise immobilize the components of the composition.

In one embodiment, other proteins or natural or synthetic materials may be substantially excluded from the hydrophilic polymer-enzyme composition of the enzyme layer. For example, the hydrophilic polymer-enzyme composition may be substantially free of bovine serum albumin. Bovine albumin-free compositions may be desirable for meeting various governmental regulatory requirements. Thus, in one embodiment, the enzyme layer comprises glucose oxidase and a sufficient amount of cross-linking agent, for example, a dialdehyde such as glutaraldehdye, to cross-link or otherwise immobilize the enzyme. In other embodiment, the enzyme layer comprises glucose oxidase, poly-N-vinylpyrrolidone and a sufficient amount of cross-linking agent to cross-link or otherwise immobilize the enzyme. In another embodiment, the enzyme layer is substantially free of redox-mediators, for example, electron-transfer mediators having compositions comprising a metal.

The enzyme layer thickness may be from about 0.05 microns or less to about 20 microns or more, more preferably from about 0.05, 0.1, 0.15, 0.2, 0.25, 0.3, 0.35, 0.4, 0.45, 0.5, 1, 1.5, 2, 2.5, 3, or 3.5 microns to about 4, 5, 6, 7, 8, 9, 10, 11, 12, 13, 14, 15, 16, 17, 18, 19, or 19.5 microns. In one embodiment, the enzyme layer is deposited by spray or dip coating, however, other methods of forming the enzyme layer may be used. The enzyme layer may be formed by dispensing methods, deposition, micro-pipetting, dip coating and/or spray coating one or more layers at a predetermined concentration of the coating solution, insertion rate, dwell time, withdrawal rate, and/or desired thickness.

### Flux Limiting Membrane Layer

In one embodiment, the flux limiting layer 25 alters or changes the rate of flux of one or more of the analytes of interest. Although the following description is directed to a flux limiting layer for an electrochemical glucose sensor, the flux limiting layer may be modified for other analytes and co-reactants as well.

In one aspect, the flux limiting layer comprises a semi-permeable material that controls the flux of oxygen and glucose to the underlying enzyme layer, preferably providing oxygen in a non-rate-limiting excess. As a result, the upper limit of linearity of glucose measurement is extended to a much higher value than that which is achieved without the flux limiting layer. In one embodiment, the flux limiting layer exhibits an oxygen to glucose permeability ratio of from about 50:1 or less to about 400:1 or more, preferably about 200:1. Other flux limiting layers may be used or combined, such as a membrane with both hydrophilic and hydrophobic polymeric regions, to control the diffusion of analyte and optionally co-analyte to an analyte sensor. For example, a suitable membrane may include a hydrophobic polymer matrix component such as a polyurethane, or polyetherurethaneurea. In one embodiment, the material that forms the basis of the hydrophobic matrix of the layer can be any of those known in the art as appropriate for use as membranes in sensor devices and as having sufficient permeability to allow relevant compounds to pass through it, for example, to allow an oxygen molecule to pass through the layer from the sample under examination in order to reach the active enzyme or electrochemical electrodes. For example, non-polyurethane type layers such as vinyl polymers, polyethers, polyesters, polyamides, inorganic polymers such as polysiloxanes and polycarbosiloxanes, natural polymers such as cellulosic and protein based materials, and mixtures or combinations thereof may be used.

In one embodiment, the flux limiting layer comprises a polyethylene oxide component. For example, a hydrophobic-hydrophilic copolymer comprising polyethylene oxide is a polyurethane polymer that includes about 20% hydrophilic polyethylene oxide. The polyethylene oxide portions of the copolymer are thermodynamically driven to separate from the hydrophobic portions (e.g., the urethane portions) of the copolymer and the hydrophobic polymer component. The 20% polyethylene oxide-based soft segment portion of the copolymer used to form the final blend effects the water pick-up and subsequent glucose permeability of the membrane.

In one embodiment, the flux limiting layer substantially excludes condensation polymers such as silicone and urethane polymers and/or copolymers or blends thereof. Such excluded condensation polymers typically contain residual heavy metal catalytic material that may otherwise be toxic if leached and/or difficult to completely remove, thus rendering their use in such sensors undesirable for safety and/or cost.

In another embodiment, the material that comprises the flux limiting layer may be a vinyl polymer appropriate for use in sensor devices having sufficient permeability to allow relevant compounds to pass through it, for example, to allow an oxygen molecule to pass through in order to reach the active enzyme or electrochemical electrodes. Examples of materials which may be used to make the flux limiting layer include vinyl polymers having vinyl ester monomeric units. In another embodiment, a flux limiting layer comprises poly ethylene vinyl acetate (EVA polymer). In other embodiments, the flux limiting layer comprises poly(methylmethacrylate-co-butyl methacrylate) blended with the EVA polymer. The EVA polymer or its blends may be cross-linked, for example, with diglycidyl ether. Films of EVA are very elastomeric, which may provide resiliency to the sensor for navigating a tortuous path, for example, into venous anatomy.

The EVA polymer may be provided from a source having a composition anywhere from about 9 wt % vinyl acetate (EVA-9) to about 40 wt % vinyl acetate (EVA-40). The EVA polymer is preferably dissolved in a solvent for dispensing into the well formed in the sensor or sensor assembly. The solvent should be chosen for its ability to dissolve EVA polymer, to promote adhesion to the sensor substrate and enzyme electrode, and to form a solution that may be effectively dispensed (e.g. micro-pipette, spray, dip coating, spin coating). Solvents such as cyclohexanone, paraxylene, and tetrahydrofuran may be suitable for this purpose. The solution may include about 0.5 wt % to about 6.0 wt % of the EVA polymer. In addition, the solvent should be sufficiently volatile to evaporate without undue agitation to prevent issues with the underlying enzyme, but not so volatile as to create problems with the dispensing process. In an embodiment, the vinyl acetate component of the flux limiting layer includes about 20% vinyl acetate. In other embodiments, the flux limiting layer is deposited onto the enzyme layer to yield a layer thickness of from about 0.05 microns or less to about 20 microns or more, more preferably from about 0.05, 0.1, 0.15, 0.2, 0.25, 0.3, 0.35, 0.4, 0.45, 0.5, 1, 1.5, 2, 2.5, 3, or 3.5 microns to about 4, 5, 6, 7, 8, 9, 10, 11, 12, 13, 14, 15, 16, 17, 18, 19, or 19.5 microns, and more preferably still from about 5, 5.5 or 6 microns to about 6.5, 7, 7.5 or 8 microns. The flux limiting layer may be deposited onto the enzyme layer, for example, by spray coating or dip coating. In one aspect, the flux limiting layer is deposited on the enzyme layer by coating a solution of from about 1 wt. % to about 5 wt. % EVA polymer and from about 95 wt. % to about 99 wt. % solvent.

In one embodiment, an electrochemical analyte sensor fabricated as described above comprises an electrode formed on an inorganic substrate, the sensor being encapsulated in a flux limiting layer covering the analyte sensing membrane layers and the underlying isolated electrode. Thus, the flux limiting layer formed from an EVA polymer may serve as a flux limiter at the top of the electrode, but also serve as a sealant or encapsulant at the enzyme/electrode boundary and at the electrode/dielectric boundary.

### Additional Membrane Layers

Additional membrane and/or other electrode layers may be deposited that provide specific functions for improving the performance of the sensor. For example, additional layers may provide for manipulation of various biological processes when used in vivo in a subject. The additional layers may provide shielding of external electrical or magnetic fields (EMF or RF). The additional layers may be adjacent to or cover at least a part of the flux limiting layer. The additional layers may include hydrophilic polymer membranes, polymers with pendent ionizable groups (polyelectrolytes) and copolymers thereof.

In one embodiment, an additional layer is a hydrophilic polymer membrane that is essentially water-insoluble. As used herein, the phase “water-insoluble” refers to a hydrophilic polymer membrane that, when exposed to an excess of water, may swell or otherwise absorb water to an equilibrium volume, but does not dissolve into the aqueous solution. As such, a water-insoluble material generally maintains its original physical structure during the absorption of the water and, thus, must have sufficient physical integrity to resist flow and diffusion away or with its environment. As used herein, a material will be considered to be water insoluble when it substantially resists dissolution in excess water to form a solution, and/or losing its initial, film form and resists becoming essentially molecularly dispersed throughout the water solution. In one embodiment, the hydrophilic polymer membrane is coated over the flux limiting layer and will not degrade or diffuse away from the flux limiting layer during use, for example, during in vivo use.

Each of the above membrane layers may be disposed over the conductive ink and/or one or more of the other electrode layers. For example, the interference layer 30 may be at least partially coated with the enzyme layer 23. Additionally, the flux limiting membrane 25 may cover the enzyme layer 23 and the interference layer 30 and at least a portion of electrode 19.

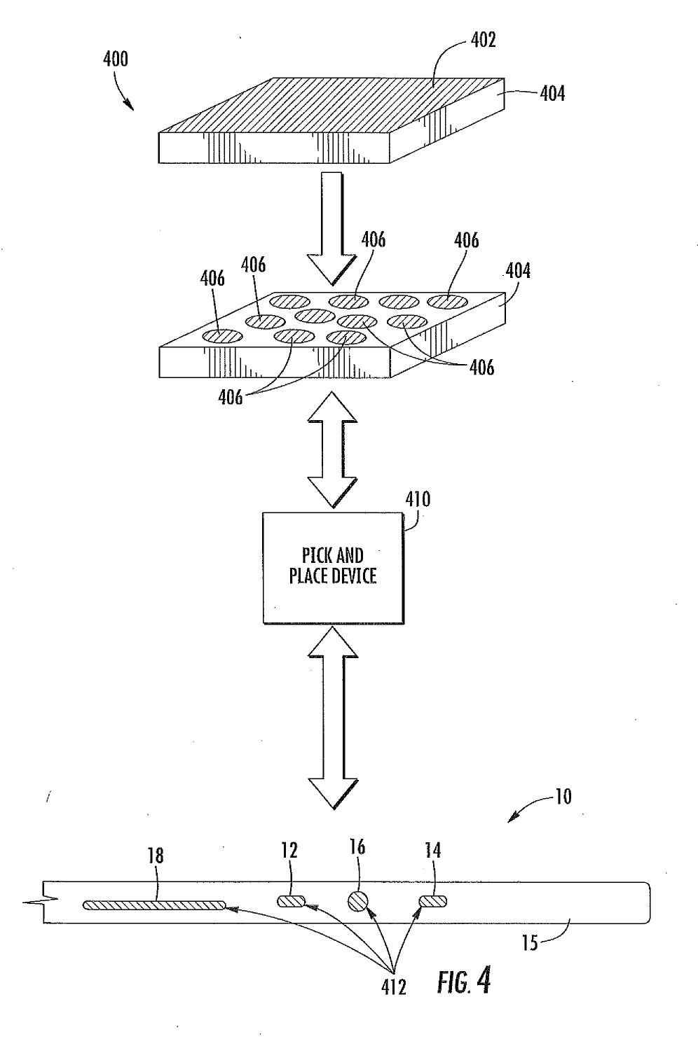

The fabrication processes and systems 200-600, described below with respect to FIGS. 2-7, detail fabrication of one or more electrodes of a biosensor by depositing conductive ink, membrane layers and/or other layers to the substrate. Specifically, processes and systems 200-500 illustrate various embodiments of depositing various layers (e.g., conductive ink, membrane layers, etc.) to a substrate in fabricating electrodes of a biosensor. Further, process 600 illustrates an embodiment of pad printing one or more layers on the substrate and/or on one or more layers already deposited on the substrate. For example, in one embodiment, membrane layers may be pad printed via the processes and systems 200-700 illustrated and described below with regard to FIGS. 2-7. In another embodiment, the processes and systems 200-700 may relate to pad printing an electrode or two or more layers to a substrate, where the electrode may include a combination of a conductive ink layer and at least one membrane layer. Each of the processes and systems 200-600 are described in more depth below.

### Fabrication Processes and Systems

Processes and systems for fabricating a biosensor will now be described with regards to FIGS. 2-7. Turning first to FIG. 2, FIG. 2 illustrates a flow chart of a biosensor fabrication process 200 for deposition of one or more layers of a biosensor electrode according to one embodiment of the present invention. As previously mentioned, the layers may include conductive ink and/or one or more membrane layers. In one embodiment, the layer is only conductive ink. In one aspect, the conductive ink has a viscosity of between about 20,000 and 30,000 centipoise (cps).

The fabrication process 200 of FIG. 2 begins in block 201 with one or more layers (e.g., conductive ink and/or membrane layers) being deposited on a surface, such as a roll of stock paper/plastic, a conveyer belt, or other surface. In one embodiment, the layers may be one or more liquid layers, where the liquid layer has a viscosity as low as a fluid and as high as paste. Individual layers may be deposited in a sequence or a single layer comprising two or more individual layers may be deposited. A deposition unit precisely places the layers on the surface. The layers may be deposited on the conveyor surface to form a plurality of individualized layer elements of a predetermined shape, such as individualized dots or disks, consisting of the one or more layers. These layer elements may be of any predefined shape or other geometrical shape. After deposition onto the conveyor, each individual layer element may be cured, thermally treated, exposed to gases or air, RF generated gas plasma, or other treatment via any other process. In one aspect, the surface receiving the layer may be heated or exposed to actinic radiation so as to rapidly treat (e.g., cure or dry) the individual layer element.

In block 202, one or more of the individual layer elements are then transferred from the surface (e.g., paper/plastic roll or conveyor) to a flexible printed circuit board using an surface-mount technology (SMT) component placement systems, sometimes referred to as a “pick-and-place machine.” The pick-and-place machine, suitable for use in the method and systems described herein, may be, for example, a robotic machine which is used to place surface-mount devices (SMDs) onto a printed circuit board (PCB), a MEMs-based substrate or other substrate. Further, the pick and place machine may be adapted for high speed, high precision placing of a broad range of electronic components, or other items, onto the PCBs and for automatically lifting, transferring and placing all deposited elements from the conveyer to a surface of the substrate (e.g., a printed circuit board). It should be understood that the pick and place device may be an automated system or one that is manually operated.

In block 203, a determination is made as to whether any deposited individual elements are left to be transferred from the conveyer to a substrate or printed circuit board. If so, then the process 200 may repeat step 202; otherwise, the process 200 may end.

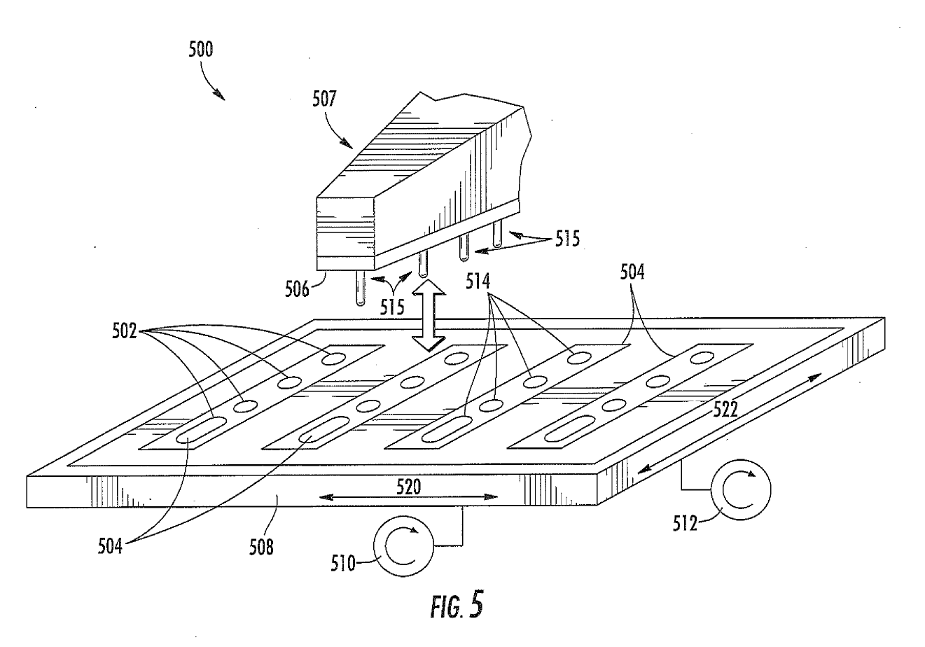

FIG. 3 illustrates an exemplary biosensor fabrication process 300 corresponding to the process 200 of FIG. 2 according to some embodiments of the present invention. As illustrated, a conveyer 302 that is capable of automated or manual movement 304 has a surface 306 upon which to receive one or more individual layer elements 308 (e.g., conductive ink dots, etc.). Two rollers 312a, 312b are connected at opposing ends of the conveyer 302. The rollers 312a, 312b rotate in a direction 314 about an axis so that the conveyer surface 306 constantly moves in a predetermined direction 304. A deposition machine 316 deposits each individual electrode elements 308 onto the conveyer surface 306. The electrode elements 308 rest on the conveyer surface 306 and may be cured, treated, exposed to air, and the like, as previous mentioned. Thereafter, the conveyer surface 306 moves the electrode elements 308 a predetermined distance and the “pick and place” machine 318 (or other device) lifts, transfers and/or releases the electrode elements 308 from the conveyor surface 306 to a substrate 15, such as flex circuit or the like.

Substrate 15 may be further fabricated using process 300 after deposition of the conductive ink onto a substrate. For example, additional layers, such as an electrode layer, interference layer, enzyme layer, flux-limiting layer and other layers for fabricating a biosensor 10, as previously discussed in FIG. 1, may be deposited in a similar manner, or optionally, in combination with conventional coating/deposition methods. For example, one or more membrane layers may be deposited on the conductive ink to form a working electrode 14 of the biosensor 10. In another embodiment, deposition of the conductive ink completes one or more electrodes of the biosensor, such as the blank or reference electrode of the biosensor 10.

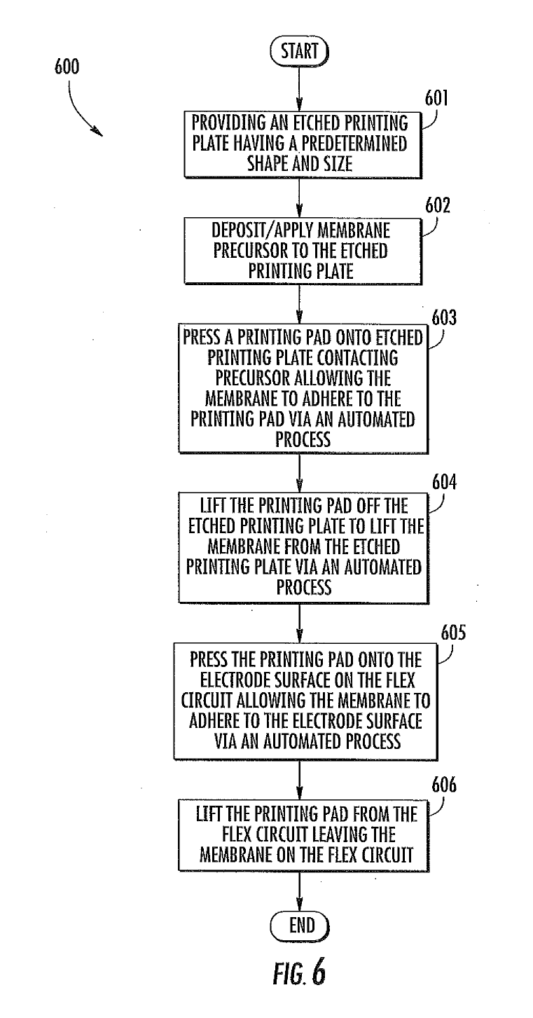

FIG. 4 illustrates another biosensor fabrication system and process 400 for depositing at least one layer onto substrate 15 of a biosensor 10. First, a sheet of one or more layers 402 (e.g., conductive ink, membrane layers, and/or other materials) is deposited onto a surface, such as a sheet substrate 404, to coat the surface. Such sheet 402 is then allowed to be treated, such as allowing the deposited layer(s) to dry onto the sheet substrate 404. Thereafter, the electrode layers sheet 402 is divided or patterned into a plurality of separate elements 406, for example, using lithographic or screen printing techniques. In one embodiment, the separated elements 406 are divided by punching or cutting out predetermined shapes (e.g., disks) of the desired shape and size. The shapes 406 are then transferred from the sheet substrate 404 and deposited onto a biosensor substrate 15 using a machine 410, such as the “pick and place” machine, previously discussed with reference to FIG. 3. The “pick and place” machine 410 may place the conductive shapes 406 on the biosensor 10, for example to fabricate one or more electrodes 12, 14, 16, and 18.

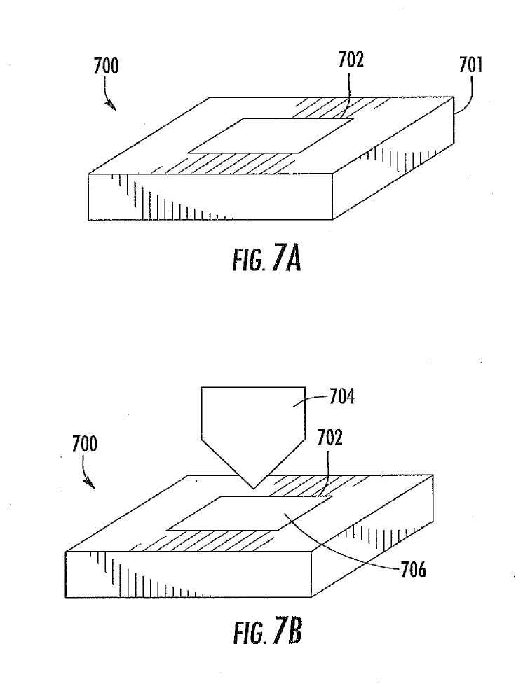

In yet another embodiment, FIG. 5 illustrates a biosensor fabrication system and process 500 to deposit one or more layers 502 directly to substrate 504. Substrate 504 may be similar to the substrate 15 discussed above in FIGS. 1a-d, for example, a flexible substrate. The system 500 of FIG. 5 includes a printing head 506, a platen 508, and adjustable controls 510, 512. The printing head 506 is generally adapted to dispense or deposit one or more layers 502 to a surface 514 of substrate 504 using one or more dispensing elements 515, as illustrated in FIG. 5. The printing head 506 may be a portion of a deposition device 507 which facilitates dispensing of the layers 502. The dispensing elements 515 of the printing 506 head allow for transfer of material (e.g., conductive ink, membrane layers, etc.) from the printing head 506 directly to the receiving surface 514.

The platen 508 is generally adapted to support one or more substrates 504, for example, printed circuit boards or the like. In one embodiment, the platen 508 is connected directly to the deposition device 507 to form a unitary printing system 500. The platen 508 is operably coupled to or connected with the adjustable controls 510, 512 for accurately positioning the substrates 504 disposed on the platen 508 underneath the printing head 506.

In one embodiment, the adjustable controls 510, 512 include a first control 510 and second control 512 to manipulate the x- and y-coordinates 520, 522 of the platen 508, respectively. This allows for accurate manipulation of the position of each of the substrates 504 prior to deposition of electrode layers 502 onto the electrode surface 514. The adjustable controls 510, 512 may be any device or machine, such as an electrical step motor (not shown) connected to a computer (not shown), so that the position of the substrate 504 and the associated sensor electrode surfaces 514 are indexed to the proper position. This adjusting of the substrate position may be an automated process controlled by the computer using computer logic with an algorithm embodied in a computer readable storage medium. The algorithm may include process 500 as well as other processes. Additionally, the printing head 506 may also be connected and controlled by the computer logic and the computer algorithm to coordinate deposition with the substrate 504 positioning.

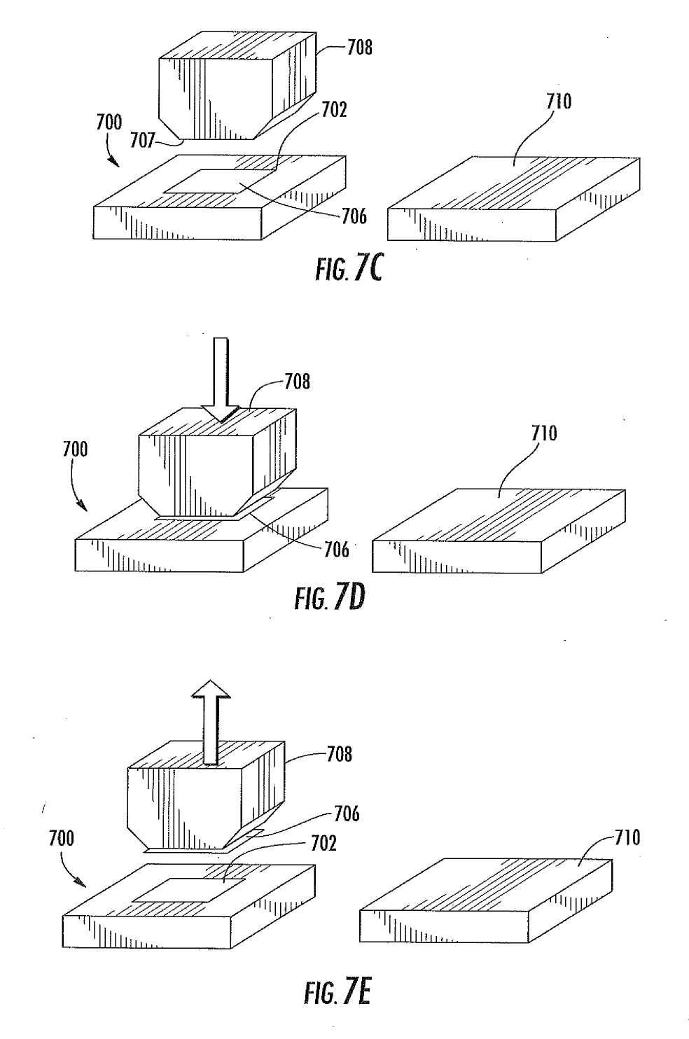

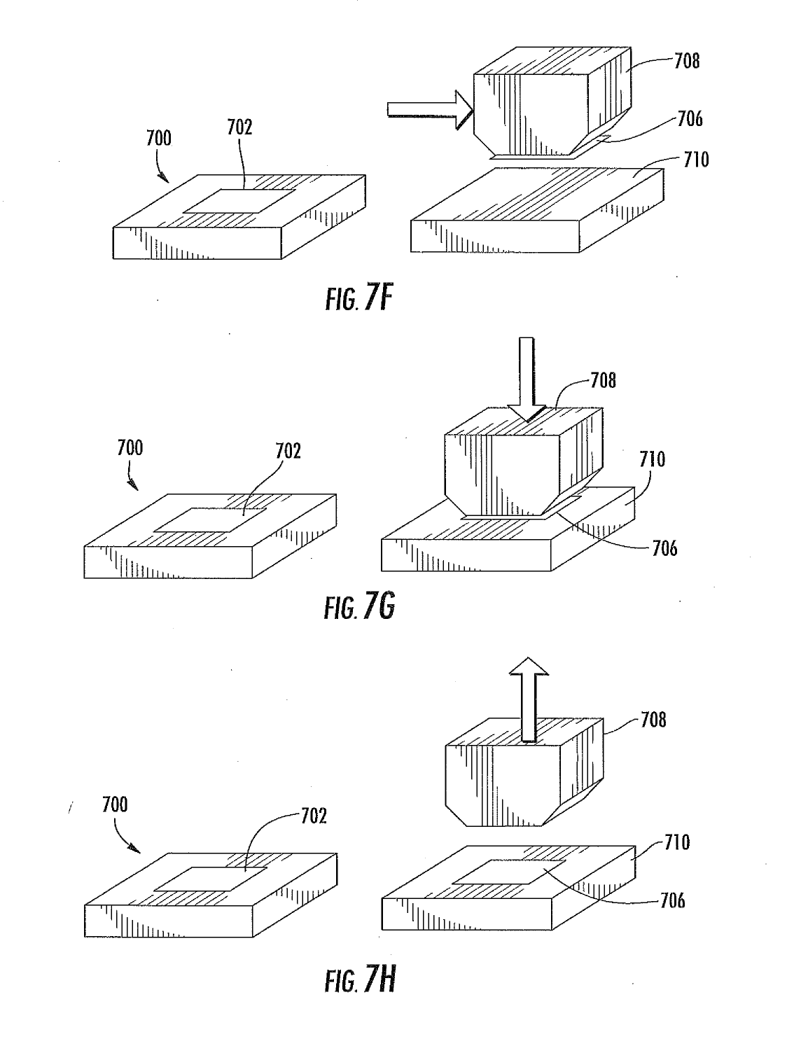

The above processes 200-500 describe fabrication of one or more layers via controlled deposition indirectly or directly onto a substrate. After depositing a layer on the substrate, another layer may be deposited thereon in forming a biosensor electrode. For example, in forming a reference electrode, after depositing a conductive material layer (e.g., a platinum-based conductive ink), one or more membrane layers or other layers (e.g, Ag or Ag/AgCl) may be deposited thereon. By way of another example, in forming a working electrode, conductive ink may be first deposited on the substrate and then membrane layers comprising an enzyme may be deposited thereon. Another exemplary method of depositing electrode layers on an intermediary surface (or directly onto the substrate) is described below with regard to process 600 and FIGS. 6-7.

FIG. 6 is a flow chart of a biosensor fabrication process 600 for the deposition of one or more layers according to one embodiment of the present invention. Elements to the pad printing system of process 600 include the printing pad, an etched printing plate, and conductive ink. In block 601, the etched printing plate is provided. The etched printing plate may be a cliche having an image of the desired membrane layer or layers etched therein. The cliché may be made of a polymer coating on a metal backing or of hardened steel. After etching such image into the etched printing plate, the etched printing plate may contain the geometrical features of the electrode layer(s) shape, diameter and thickness to be deposited and transferred to the substrate. In some embodiments, these elements may all function to work together in an automated fashion so that no human intervention is needed. It should be understood, however, that one or more of these elements may be performed or function manually. While the above description provides a high level description of FIG. 6, a more detailed description of each block of FIG. 6 is described below. It is noted that FIGS. 7A-7I are referenced while detailing the process of FIG. 6 since FIGS. 7A-7H illustrate the disclosed process of FIG. 6 according to one embodiment of the present invention.

First, in block 601 of FIG. 6, an etched printing plate is provided having a predetermined shape and size. In FIG. 7A, an exemplary etched printing plate 700 is illustrated having a cliché 701 and an electrode image 702 etched in the etched printing plate 700.

In block 602 of FIG. 6, a predetermined amount of electrode layer(s) ingredients, including one or more precursor liquids and modifier group materials, are applied to the base of a reservoir that feeds the etched printing plate. Although these electrode layer ingredients may be applied to the reservoir feeding the etched printing plate, the electrode layer ingredients may be transferred to the etched printing plate in any other manner. For example, as illustrated in the exemplary embodiment of FIG. 7B, the electrode layer ingredients 704 may be applied directly to the etched printing plate 700 to form one or more electrode layers 706. Nonetheless, in one embodiment, after deposition of the electrode layer ingredients 704 into the etched printing plate 700, the electrode layer(s) 706 may be cured, dried and/or exposed to air to harden and/or become somewhat tacky (or have some other attribute). This allows for the electrode layer(s) 706 to easily adhere to surface 707 of printing pad 708 upon contact therewith and to facilitate lift-off from etched printing plate 700.

In block 603 of FIG. 6, a printing pad is pressed onto the etched printing plate, contacting the layers (e.g., membrane layers) so as to releasably adhere to the printing pad. The printing pad may be made of any material, such as silicon, which can vary in durometer (or hardness). The properties of the silicon may be adjusted mechanically or chemically to allow the layers to temporarily stick to the pad, yet fully release from the pad when it comes into contact with the surface to be deposited on. The durometer of the pad may dictate how the image molds to the target surface of the substrate. For example, to deposit an electrode on a printed circuit board, a harder pad will deposit more of the electrode image into the textured surface. Likewise, a larger image to be applied on a flat (or nearly flat) surface would normally require a substantial amount of downward pressure to deposit the entire layer with a hard pad. By using a softer pad, the layer can be deposited using less pressure and thus avoiding complications associated with too much pressure. Regardless, as the pad is compressed on the layer(s), it may push air outward and cause the layer(s) to temporarily attach to the pad so that the layer(s) can be lifted from the etched printing plate. Additionally, the top portion of the layer(s) may be somewhat tacky due to being incompletely or semi-dried, exposed to air and/or being cured or otherwise conditioned, further facilitating the layer(s) temporarily adhering to the printing pad. FIG. 7C illustrates the layer(s) 706 positioned over the etched printing plate 700 and FIG. 7D illustrates the printing pad 708 pressing onto the layer(s) 706 such that the layer(s) 706 adheres temporarily to the pad 708.

As described in block 604 of FIG. 6 and illustrated in FIG. 7E, the printing pad 708 lifts the layer(s) 706 from the etched printing plate 700 a predetermined distance. The layer(s) 706 remains temporarily adhered to the pad 708 after being lifted off the plate 700. The printing pad 708, along with the attached layer(s) 706, is then transferred from the etched printing plate 700 to an area proximate to substrate 710, such as a flex circuit or the like as shown in FIG. 7F.

As described in block 605 of FIG. 6 and illustrated in FIG. 7G, the printing pad compresses down onto the substrate, transferring the layer(s) lifted from the etched printing plate 700 to an surface on the substrate 710, for example, to fabricate an electrode. Then, in block 606 of FIG. 6 and FIG. 7H, the printing pad 708 lifts off the substrate 710, returning to its original position, which completes one layer(s) deposition cycle. After deposition of the first layer(s) 706, one or more layers (not shown) may be deposited thereon by repeating blocks 610-606.

It should be noted that any or all of the steps 601-606 of FIG. 6 may be performed via an automated process and system. For example, the pad 708 and etched printing plate 700 may be attached to an automated machine (not shown) to perform one or more or all steps of method 600. As such, it should be appreciated that no human intervention need be required to perform one or more steps of method 600. This allows the layer(s) 706 to be applied in a constant and time efficient manner. However, it should be understood that one or more of the steps 601-606 may be performed manually and need not be limited to automated processing.

Additionally, the above-described process and system 600-700 may transfer any number of layers or other materials in any processing order or fashion. For example, in one embodiment, each electrode may be deposited on the substrate and transferred one-by-one from the etched printing plate to the substrate. In another embodiment, two or more of the layers may be deposited in the etched printed plate and thereafter transferred from the etched printing plate to the substrate. In yet another embodiment, one or more layers, such as membrane layers deposited on a conductive ink layer, may be deposited in the etched printing plate and then transferred simultaneously from the etched printing plate to the substrate via the above-described process and system 600-700.

While certain exemplary embodiments have been described and shown in the accompanying drawings, it is to be understood that such embodiments are merely illustrative of and not restrictive on the broad invention, and that this invention not be limited to the specific constructions and arrangements shown and described, since various other changes, combinations, omissions, modifications and substitutions, in addition to those set forth in the above paragraphs, are possible. Those skilled in the art will appreciate that various adaptations and modifications of the just described embodiments can be configured without departing from the scope and spirit of the invention. Therefore, it is to be understood that, within the scope of the appended claims, the invention may be practiced other than as specifically described herein.

1-56. (canceled)

57. A method of fabricating a biosensor comprising:
depositing one or more layers on a surface, the surface being releasable of the one or more layers; and
transferring the one or more layers from the surface to at least one biosensor substrate.

58. The method of claim 57, wherein the transferring is performed using a pick and place device.

59. The method of claim 57, wherein the one or more layers comprises conductive ink, optionally in combination with an enzyme-containing layer.

60. The method of claim 57, further comprising treating the one or more layers after deposition.

61. The method of claim 57, wherein the biosensor substrate comprises one of a printed circuit board or a MEMs-based substrate.

62. The method of claim 57, further comprising:
forming a sheet of the one or more layers on a surface; and
forming at least one object having a predetemined shape from the sheet.

63. The method of claim 62, wherein the at least one predetermined shaped object is a conductive material capable of forming an electrode.

64. The method of claim 57, wherein the depositing and transferring is performed by pad printing.

65. The method of claim 64, wherein the pad printing process comprises:
providing an etched printing plate and a corresponding printing pad;
introducing the one or more layers to the etched printing plate;
contacting the one or more layers with the printing pad; and
transferring the one or more layers to the at least one biosensor substrate using the printing pad.

66. The method of claim 57, wherein the one or more layers comprise at least one or more of an electrode layer, an interference layer, an enzyme layer, a flux-limiting layer, or any combination thereof.

67. The method of claim 65, wherein the pad printing one or more liquid membrane layers comprises:
applying at least one liquid membrane layer;
contacting a pad onto the at least one liquid membrane layer; and
transferring the at least one liquid membrane layer to the substrate.

68. The method of claim 57, wherein said depositing comprises:
providing a position-controlled platen for receiving at least one substrate, each of the at least one substrate having at least one surface area portion adapted for receiving an electrode-forming material;
positioning the platen in a predetermined position relative to a deposition unit; and
depositing at least one layer on the at least one surface area portion of the at least one substrate.

69. The method of claim 68, wherein the depositing comprises depositing the at least one layer at a plurality of portions on a substrate to form a plurality of electrodes on the biosensor.

70. The method of claim 68, wherein the at least one layer comprises a conductive ink and at least one membrane layer.

71. A method for fabrication of a biosensor comprising:
depositing a first working electrode at a first portion of a substrate, comprising:
depositing a first conductive ink layer on a surface; and
transferring the first conductive ink layer from the surface to the first substrate portion;
depositing a second working electrode at a second portion of the substrate, comprising:
depositing a second conductive ink layer on the surface;
depositing at least one membrane layer over the second conductive ink layer; and
transferring the second conductive ink layer from the surface to the second substrate portion; and
depositing a reference electrode at a third portion of the substrate, comprising.
depositing a third conductive ink layer on the surface; and
transferring the third conductive ink layer from the surface to the third substrate portion,
wherein one or more of the transferring steps is performed using a printing pad process or a pick and place device.

72. The method of claim 71 further comprising:
depositing a counter electrode at a fourth portion of the substrate, comprising:
depositing a fourth conductive ink layer on the surface; and
transferring the fourth conductive ink layer from the surface to the fourth substrate portion.

73. The method of claim 71, further comprising depositing conductive ink on the surface at two additional portions of the substrate to form a thermistor on the substrate.

74. The method of claim 71, further comprising:
applying at least one liquid membrane layer;
contacting a pad onto the at least one liquid membrane layer; and
transferring the at least one liquid membrane layer to the substrate on one of said first or second working electrodes.

75. A biosensor formed by a process comprising:
depositing one or more layers on a surface, the surface being releasable of the one or more layers; and
transferring the one or more layers from the surface to at least one biosensor substrate.

76. A biosensor formed by a process comprising:
depositing a first working electrode at a first portion of a substrate, comprising:
depositing a first conductive ink layer on a surface; and
transferring the first conductive ink layer from the surface to the first substrate portion;
depositing a second working electrode at a second portion of the substrate, comprising:
depositing a second conductive ink layer on the surface;
depositing at least one membrane layer over the second conductive ink layer; and
transferring the second conductive ink layer from the surface to the second substrate portion; and
depositing a reference electrode at a third portion of the substrate, comprising.
depositing a third conductive ink layer on the surface; and
transferring the third conductive ink layer from the surface to the third substrate portion,
wherein one or more of the transferring steps is performed using a printing pad process or a pick and place device.

77. A system for fabricating a biosensor, the system comprising:
a deposition device for depositing one or more layers on a surface; and
a pick and place device for transferring the one or more layers from the surface to a biosensor substrate.

78. The system of claim 77, wherein the one or more layers comprises at least one or more of an electrode layer, an interference layer, an enzyme layer, a flux-limiting layer, or any combination thereof.

79. The system of claim 77, wherein the pick and place device and the deposition device are cooperatively configured.

80. The system of claim 77, further comprising treating the one or more layers after deposition.

81. The system of claim 77, further comprising a device to cut the one or more layers into one or more predetermined shapes on the surface.

82. The system of claim 77, wherein the deposition unit is configured to deposit a plurality of predetermined shaped objects from the surface to a plurality of biosensor substrates.

83. The system of claim 77, further comprising a pad printing system to pad print one or more layers, wherein the pad printing system comprises:
an etched printing plate to receive the one or more layers; and
a printing pad adapted to contact and releasably transfer the one or more layers to the substrate.
